(19) United States
(12) Patent Application Publication
(10) Pub. No.: US 2015/0182382 A1
(43) Pub. Date: Jul. 2, 2015

(54) PRE-LUBRICATED EARPLUGS AND THE METHOD OF PRODUCTION THEREOF

(71) Applicant: Custom Protect Ear, Inc., Surrey (CA)

(72) Inventor: Alexei Bobyrev, Surrey (CA)

(73) Assignee: Custom Protect Ear, Inc., Surrey (CA)

(21) Appl. No.: 14/576,895

(22) Filed: Dec. 19, 2014

Related U.S. Application Data
(60) Provisional application No. 61/921,080, filed on Dec. 27, 2013.

(51) Int. Cl. A61F 11/08; B05D 1/18

(52) U.S. Cl. CPC A61F 11/08; B05D 1/18; A61F 2240/001

(57) ABSTRACT

A method of producing a material which seeps a desired coating for example a method of producing a self-lubricating earplug by submerging an earplug in a lubricant bath for a selected period of time for at least one of a bath temperature and/or pressure.

### CROSS-REFERENCE TO RELATED APPLICATIONS

This application claims the priority benefit under 35 U.S.C. §119(e), of U.S. Provisional Patent Application No. 61/921,080 filed on 27 Dec. 2013, incorporated herein by reference in it's entirety.

### FIELD OF THE INVENTION

The invention relates in general to hearing protection and more particularly to method of producing self lubricating devices insertable into cavities, producing pre-lubricating earplugs and other devices insertable into the human ear canal.

### BACKGROUND OF THE INVENTION

Newly made earplugs constructed of silicone, resins, acrylics, and other materials usually have a very dry surface. During a plug's insertion into a human ear such surface creates significant friction with the skin inside the ear, which at a minimum causes discomfort, and at maximum—skin abrasions. For this reason all custom hearing protection manufacturers provide their clients with a lubricant of some sort to mitigate the “new silicone” effect during the brake-in period. Another solution is to coat the silicone surface with a thin layer of a slippery material, for example Slick-Sil^{TM}. The earwax, also known by the medical term cerumen, is a yellowish waxy substance secreted in the ear canal of humans and other mammals, which slowly saturates the silicone surface of the plug making it slippery and opaque. During the first 7-10 days of wearing a new plug this process modifies the silicone surface from dry to slippery and oily. Another evidence of the absorbing process is that protectors that have been worn extensively for more than about 3 or 4 months do not coat well when layered with a silicone compound because they have absorbed body oils and continue to “sweat” even after washing.

### BRIEF DESCRIPTION OF THE DRAWINGS

Embodiments of present invention will become more fully understood from the detailed description and the accompanying drawings, wherein:

FIG. 1 illustrates general physiology of an ear;

FIG. 2A illustrates a cartilaginous region and a bony region of an ear canal;

FIG. 2B illustrates a cartilaginous region and a bony region of an ear canal with a molded earplug inserted;

FIG. 3 illustrates method of pre-lubricating according to an embodiment.

FIG. 4A illustrates an earplug submerged into a bath of lubricant in accordance to at least one embodiment;

FIG. 4B illustrates an earplug submerged into a bath of lubricant in accordance to at least one embodiment;

FIG. 4C illustrates an earplug submerged into a bath of multiple lubricants in accordance to at least one embodiment;

FIG. 5 illustrates an image of the mass increase of a self lubricating earplug according to an embodiment;

FIG. 6 illustrates an embodiment of a self lubricating earplug with lubricant seeping from the earplug;

FIG. 7 illustrates an embodiment of a self lubricating earplug inserted into an ear canal illustrating different seepage rate for different areas of contact;

FIG. 8 illustrates an embodiment of a self lubricating earplug in accordance with at least one embodiment;

FIG. 9 illustrates an embodiment of a self lubricating earplug in accordance with at least one embodiment; and

FIG. 10 illustrates an embodiment of a self lubricating hearing aid in accordance with at least one embodiment.

### DETAILED DESCRIPTION OF EMBODIMENTS

The following description of embodiment(s) is merely illustrative in nature and is in no way intended to limit the invention, its application, or uses.

For simplicity and clarity of the illustration(s), elements in the figures are not necessarily to scale, are only schematic and are non-limiting, and the same reference numbers in different figures denote the same elements, unless stated otherwise. Additionally, descriptions and details of well-known steps and elements are omitted for simplicity of the description. Notice that once an item is defined in one figure, it may or may not be discussed or further defined in the following figures.

It will be appreciated by those skilled in the art that the words “during”, “while”, and “when” as used herein are not exact terms that mean an action takes place instantly upon an initiating action, rather these terms refer to an action where there may be some small but reasonable delay, such as a propagation delay, between the reaction that is initiated by the initial action. Additionally, the term “while” means that a certain action occurs at least within some portion of duration of the initiating action. The use of the word “approximately” or “substantially” means that a value of an element has a parameter that is expected to be close to a stated value or position. However, as is well known in the art there are always minor variances that prevent the values or positions from being exactly as stated.

The terms “first”, “second”, “third” and the like in the Claims or/and in the Detailed Description are used for distinguishing between similar elements and not necessarily for describing a sequence, either temporally, spatially, in ranking or in any other manner. It is to be understood that the terms so used are interchangeable under appropriate circumstances and that the embodiments described herein are capable of operation in other sequences than described or illustrated herein.

Processes, techniques, apparatus, and materials as known by one of ordinary skill in the art may not be discussed in detail but are intended to be part of the enabling description where appropriate. For example, specific methods of attaching an earplug for submergence into a vat are not discussed nor methods for cleaning an earplug which are known by one of ordinary skill in the arts. Thus, one of ordinary skill would be able, without undo experimentation, to establish the steps using the enabling disclosure herein.

The terms precision and resolution can be used herein to specifically have the standard definitions. Precision will connate the variation from exactness. Resolution will have the customary definition of the smallest measurable interval.

Note that discussion herein refers to an earplug or other ear insert device and lubrication for ease of insertion, however the process can be used on any device for various objectives where gradual seepage or self lubrication is desired, for example to increase the useful lifetime of any device that would dry out over time (e.g., via UV exposure), a device that when inserted into a cavity can have the sealing enhanced by gradual seepage, a device where friction is reduced by the gradual seepage,

In the enabling description herein silicone is referred to although any material that can absorb lubricant can be used and the discussions herein should not be interpreted to limit the material used. Silicone material used in the earplugs is hydrophobic, however, it can moderately absorb substances that it is submerged in. Alternative embodiments can use hydrophilic materials.

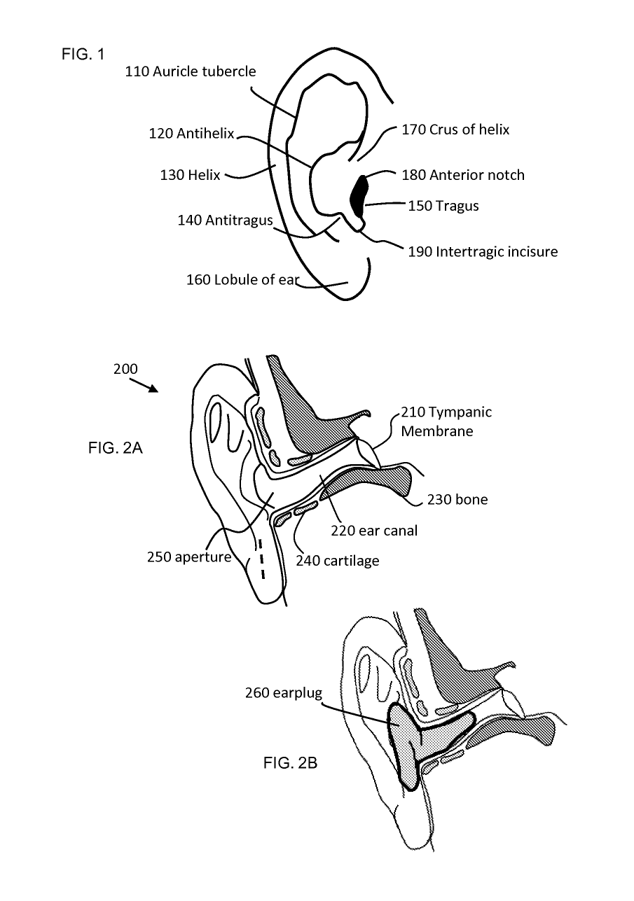

FIG. 1 illustrates general outer physiology of an ear, which includes a, auricle tubercle 110, the antihelix 120, the helix 130, the antitragus 140, tragus 150, lobule of ear 160, crus of helix 170, anterior notch 180, and intertragic incisures 190.

FIG. 2A illustrates a generic cross section of an ear canal 200, including a cartilaginous region 240 and a bony region 230 of an ear canal 220. The entrance of the ear canal 220 is referred to as the aperture 250 and defines a first end of the ear canal while the tympanic membrane 210 defines the other end of the ear canal 220.

FIG. 2B illustrates a generic cross section of an ear canal with an inserted earplug 260. Earplugs are discussed herein and materials and styles of manufacture are known by one of ordinary skill in the arts, for example: foam, silicone, and resin materials, and flanged, unflanged, pre-molded, or custom-molded styles. Discussion herein of particular type of earplug should not be interpreted to limit the scope of the invention.

### AT LEAST ONE NON-LIMITING EXEMPLARY EMBODIMENT DESCRIPTION

The hearing protection pre-lubrication process is intended to emulate the above natural absorption process by saturating the surface of the earplug with a specially selected lubricant (e.g., mineral oil, baby oil, sweet almond oil). The method according to at least one embodiment accelerates the natural process. The method thus reduces or eliminates the need in using an additional lubrication during the break-in period of a new earplug. The lubricant can have specific properties that allow it to be released to the surface of the pre-treated earplug at a certain rate and providing the necessary lubrication. After a period of time, the lubricant in the surface of an earplug will ultimately be replaced by the cerumen in most individuals.

The commercial advantage of the process over conventional systems is an approach that addresses the long term problem of lubricating the silicone hearing protectors. In the described process and system herein the selected lubricant remains in the earplug material (e.g., silicone) for an extended period of time, and the lubricant releases at a desired rate, where, when released provides sufficient lubrication. Note that simply submerging a silicone plug in a lubricant will not produce the desired effect as the natural surface saturation process is very slow. Tuning the process, as described herein, allows the lubricant to saturate the surface within a short period of time adding measurable mass to the treated earplug.

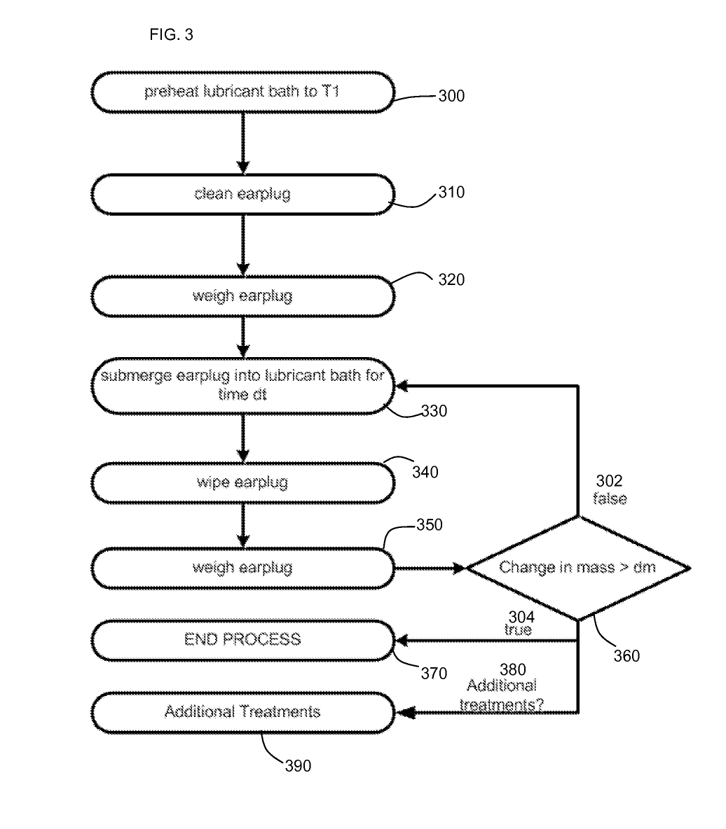

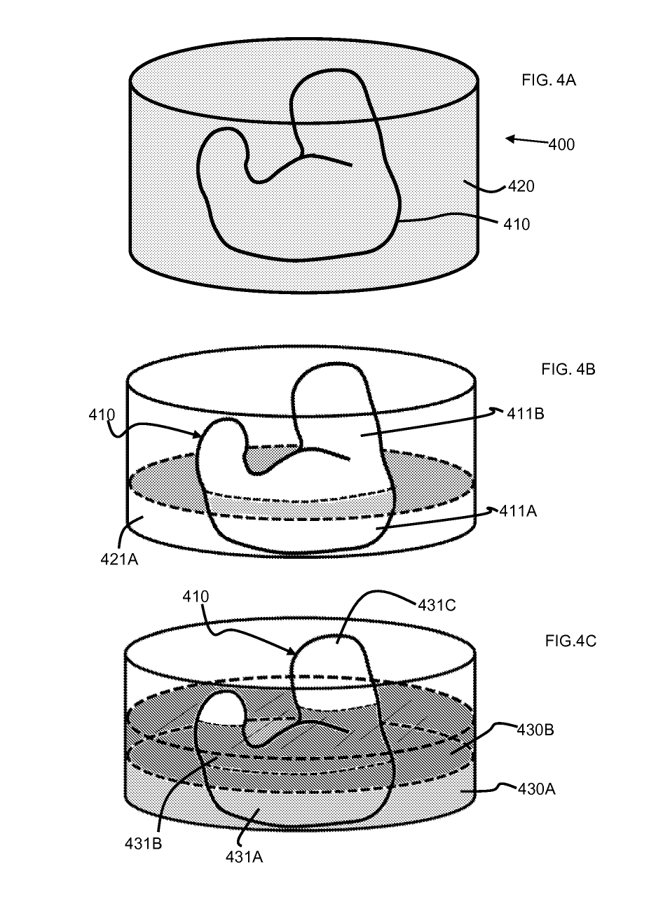

FIGS. 3 and 4A illustrates a non-limiting example of a process and apparatus respectfully to pre-lubricate a hearing protection system (e.g., earplug) in accordance with at least one embodiment. FIG. 4A illustrates 400 a non-limiting example of a molded silicone earplug 410 submerged in a heated lubricant bath 420 for a predetermined time. The lubricant (e.g., food grade mineral oil, more specifically an oil that has in general Specific Gravity at 25 Deg C./25 Deg C. ASTM D4052 0.800/0.900, Kin. Viscosity at 40 Deg C., mm2/s ASTM D445 10/20, or in general any type of oil that can be absorbed into the material of the earplug) bath is preheated 300 to a temperature T1 (operating temperature, for example 90 F to 500 F, or more specifically 100 F to 300 F). Note that the system in FIG. 4A can be sealed and the lubricant pressurized>1 atm to aid in the forcing of the lubricant into the earplug 410. The earplug 410, prior to submergence can be cleaned 310 (e.g., with detergent or just by dry wiping). The earplug 410 is then submerged 330 in a lubricant bath, for example submerged in a bath of hot mineral oil for set period of time dt (e.g.,0.10 to 5 hours, or more specifically 0.5 h to 3 h) to achieve the necessary level of the surface saturation. After treatment the earplugs can be wiped/blown dry 340 and weighed 350 to get the post treatment weight. To maintain quality control one can obtain the initial weight of the pre-treated earplug 320 then compare to after treatment to make sure the increase in mass, dm, is above a selected reference value of change of mass or a percentage increase over the start mass. For example the change in mass dm can fall within a range, dmₘᵢₙ<dm<dmₘₐₓ. Thus, the post treatment weight can be compared 360 with the target value of change of mass or percentage increase; for example, within a mass increase range from 0.1 g (dmₘᵢₙ) to 1.5 g (dmₘₐₓ), or more specifically 0.3 g to 0.5 g depending on the size of the plug. If the increase in mass falls 304 within the mass increase range or greater than a target value then the process can end 370 or a check 380 to determine if additional treatments 390 is desired. If the increase in mass is 302 not within the predetermined range or not greater than the value set, then the earplug can be returned to the lubricant bath 330. Additional treatments 390 (e.g., FIGS. 4B and 4C) can add other characteristics, for example, a different submergence oil at a second temperature, for a second period of submergence time can follow the first treatment to add additional characteristics (e.g., increase self lubrication at body temperatures). Note that a lubricant can also be used to establish particular property of the earplug. For example to reduce the freezing of an earplug in cold climates particular lubricants can be chosen for the submergence treatment. Additionally suppose a florescent earplug is needed for night time use, or for ease of location in dark environments. A fluorescent lubricant can be used, or a florescent fluid not a lubricant but which can also be absorbed, for example U.S. Pat. No. 6,749,772 by William Zumdome, incorporated herein in its entirety, describes a fluorescent lubricant. Additional treatments can include: washing the earplug to degrease the surface (e.g., hot soapy water); and applying a topcoat layer (e.g., silicone) within a period of time of washing (e.g., 5 minutes), where the topcoat layer is chosen to aid in controlling lubricant release. For example the thickness of the topcoat layer can be varied to control the lubricant seeping from the earplug through the topcoat layer. Additionally the composition of the topcoat can be varied to control the seepage rate.

FIG. 4B illustrates an earplug submerged into a bath of lubricant in accordance to at least one embodiment. In this particular non-limiting example a first part of the earplug 411A is submerged 421A, while a second part 411B is not submerged. Note that in a subsequent submergence the second part 411B can be submerged in a separate process, for example a different lubricant under different or the same conditions. In at least one embodiment, an earplug 410 is submerged in a first lubricant to a first level 411A. This provides absorption of the first lubricant in region 411A. After the predetermined soakage time and temperature and/or pressure the earplug 410 is removed and submerged in a second lubricant to different level. The previous level of absorption of the first lubricant reduces the absorption level of the second lubricant in the previously regions exposed to the first lubricant. The result is that most of the second lubricant is absorbed in the regions not submerged in the first lubricant but submerged in the second lubricant, while the previous regions submerged in the first lubricant are saturated with the first lubricant reducing the second lubricant absorption in those regions. Using additional submergences one can absorb different lubricants in different regions of the earplug 410.

FIG. 4C illustrates an earplug 410 submerged into a bath of multiple lubricants (430A and 430B) in accordance to at least one embodiment. In this non-limiting example one lubricant 430A can have a higher specific gravity than the second lubricant 430B, where the first part of the earplug 431A is submerged in the first lubricant 430A, while the second part of the earplug 431B is submerged in the second lubricant 430B. In this example a third part 431C of the earplug 410 is not submerged.

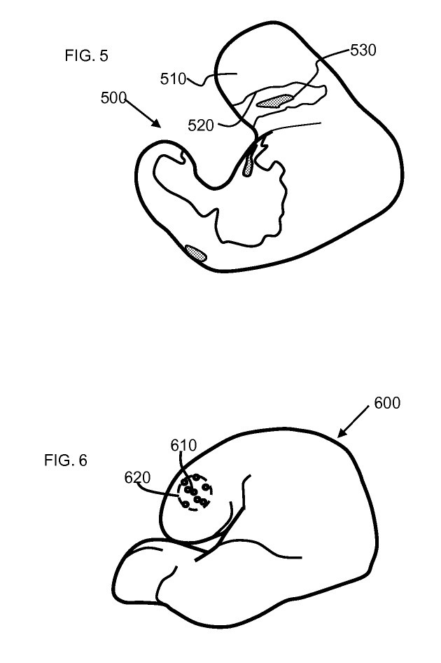

FIG. 5 illustrates 500 a contour illustration of the various regions 520 and 530 of the earplug 510 and the associated post-treatment increases in mass. For example region 530 can represent a larger lubricant mass absorption in region 530, with less in region 520 and still less in comparison in region 510. Thus various regions of the earplug can contain more increase in absorbed mass. Therefore various regions can also seep at different designed rates depending upon different exposure conditions. Alternatively as discussed above multiple submergences can result in different regions of the earplug seeping different lubricants in different regions. Additionally, the seepage rate may be the same in the various regions. The seepage rates can be manipulated such that they vary in different conditions, for example variations in temperature, pH, and humidity. The seepage rate can have various values, for example is between 0.025 mg/cm2 per week and 0.040 mg/cm2 per week, or from 3-10% of the absorbed lubricant seeping over a period of about a year. The seepage can seep through various surface areas of an earplug for example a typical surface area is about 1800 mm² with a standard deviation of about 215 mm². For example a typical value of absorbed lubricant is 22.34 mg/cm2 and ranges from about 20.5 mg/cm2 (for large plugs) to 26.2 mg/cm2 (for small plugs). In at least one embodiment a self lubricating earplug in accordance with at least one embodiment that has been exposed to cold weather can have seepage rate increased by exposing the earplug to an elevated temperature (e.g., body temperature) for an exposure time (e.g., 1 minute).

FIG. 6 illustrates an embodiment of a self-lubricating earplug 600, where beads 610 of lubrication seeps from the earplug (hearing protection) material (e.g., foam, silicone) from a designed seepage area 620. Note that the designed seepage area can be the entire earplug or a select portion of the earplug. The self-lubrication is made possible by the pre-lubrication process as described herein, for example FIGS. 3 and 4A, 4B, and 4C.

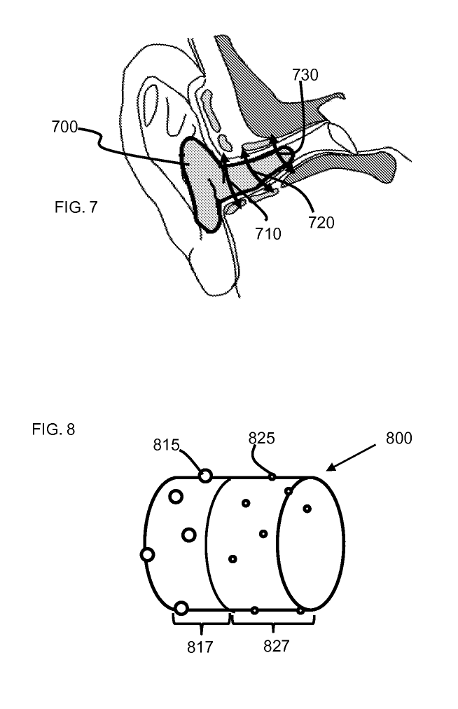

FIG. 7 illustrates an embodiment of a self lubricating earplug inserted into an ear canal illustrating different seepage rate for different areas of contact. For example an earplug 700, processed as described herein such that various seepage rates, seeps at different rates 710, 720, and 730 depending upon which location the earplug sits in the ear canal. For example the region of the earplug associated with seepage rate 730 may have been a partial submergence for a longer time than a partial submergence of the region of the earplug associated with seepage 710. For example more seepage might be needed in location related to seepage 720.

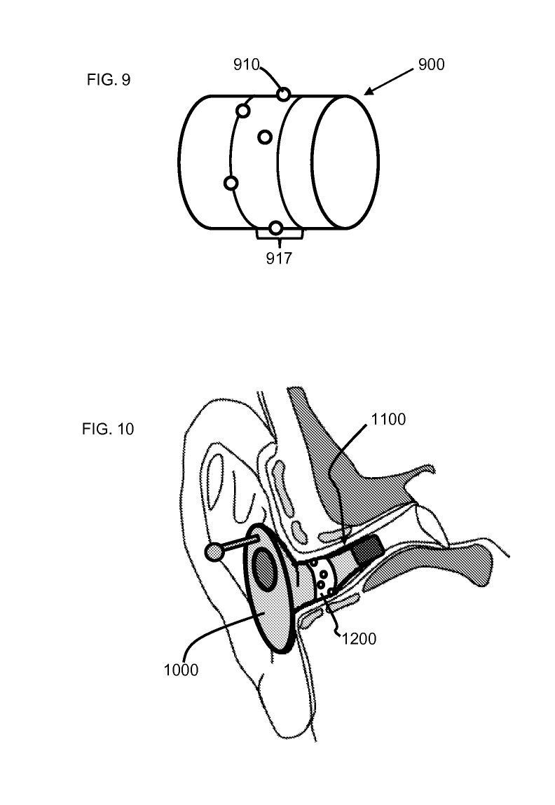

FIG. 8 illustrates an embodiment of a self lubricating earplug 800 in accordance with at least one embodiment. In this non limiting example a non molded earplug 800 (e.g., foam) has been treated such that two different regions 817 and 827 have been treated differently resulting in different seepage rates and/or lubricants. For example region 817 can seep more 815 than the seepage 825 in region 827. At least one additional embodiment can coat the non-molded earplug 800 with a topcoat layer to control lubricant seepage. In yet another embodiment the topcoat layer can be treated with a different self lubricant that interacts with the lubricant used to originally treat regions 817 and 827.

FIG. 9 illustrates an embodiment of a self lubricating earplug 900 in accordance with at least one embodiment. In this particular non-limiting example a portion 917 of the earplug seeps lubricant 910, while the rest of the earplug seepage is at a substantially reduced rate. Note that the method can be used in other devices such as hearing aids. Additionally note that portion 917 can be a strip that is treated separately then adhered to earplug 900.

FIG. 10 illustrates a hearing aid 1000 inserted into an ear canal 1100 with a region 1200 that has been treated by a process in accordance with at least one embodiment. For example the hearing aid can be fabricated from a first medium, then a second more absorptive layer 1200 (e.g., silicone) can be submerged in the lubricant and attached later, or the shell of the hearing aid can be submerged, where the layer absorbs the lubricant while the rest less so. The absorptive later 1200 will then seep lubricant to aid sealing of the hearing aid and to aid insertion. Note that additionally a strip or layer can be treated separately then attached as part of the hearing aid or headphone.

While the present invention has been described with reference to embodiments, it is to be understood that the invention is not limited to the disclosed embodiments. The scope of the following claims is to be accorded the broadest interpretation so as to encompass all modifications, equivalent structures and functions. Additionally although specific numbers may be quoted in the claims, it is intended that a number close to the one stated is also within the intended scope, i.e. any stated number (e.g., 90 degrees) should be interpreted to be “about” the value of the stated number (e.g., about 90 degrees).

Although enabling non limiting examples describe earplugs , hearing aids/headphones, the technique described can be applied to any object or device where lubrication or protective coating are useful. For example surfaces of devices where exposure to UV could damage the surface. In such a case the lubricant could be UV blocking. For example exposed cladding of wires could be treated with a UV protective lubricant that gradually seeps out of the cladding keeping the wire cladding from drying out. Likewise buried pipes could be treated to be resistance to erosion or reacting with either the external environment or even the interior of the piping can be treated to seep a lubricant or coating that aids in resistance of the interior pipe reacting with the carried fluids. As the claims hereinafter reflect, inventive aspects may lie in less than all features of a single foregoing disclosed embodiment. Thus, the hereinafter expressed claims are hereby expressly incorporated into this Detailed Description of the Drawings, with each claim standing on its own as a separate embodiment of an invention. Furthermore, while some embodiments described herein include some but not other features included in other embodiments, combinations of features of different embodiments are meant to be within the scope of the invention, and form different embodiments, as would be understood by those skilled in the art.

What is claimed is:

1. A method of producing a self-lubricating hearing protection system comprising:
cleaning a hearing protection system; and
submerging the hearing protection system into a heated lubricant bath, where the lubricant bath is a temperature T1 within a range of 100 F to 300 F, for a period of time within a range 0.5 to 3 hours.

2. The method according to claim 1, where the lubricant is mineral oil.

3. The method according to claim 1, where the lubricant has a specific gravity between 0.7 to 0.9.

4. The method according to claim 1, where the lubricant has a kinematic viscosity at 40 degrees C. between 10 to 20 mm̂2/s.

5. The method according to claim 2, further comprising:
weigh the hearing protection system prior to submergence to obtain the pretreatment weight.

6. The method according to claim 5, further comprising:
wiping the hearing protection after submergence.

7. The method according to claim 6, further comprising:
weighing the hearing protection system after submergence to obtain the after treatment weight.

8. The method according to claim 7, further comprising:
subtract the pre treatment weight from the after treatment weight to obtain a weight change value; and
comparing the weight change value within a predetermined mass change range, and if the weight change value is less than the lower value of the mass change range then the earplug is resubmerged for a time period dt2.

9. The method according to claim 8 where the mass change range is from 0.1 g to 1.5 g.

10. The method according to claim 9, further including:
submerging the earplug a second time after the first submergence, where the earplug is submerged a second time for a period of time between 0.25 and 3 hours into a second lubricant.

11. A self-lubricating earplug comprising:
a material conformable to fit within an ear canal; and
a lubricant absorbed within the material at a treatment temperature for a treatment time, where the lubricant seeps out at a predetermined rate at body temperature.

12. The earplug according to claim 11, where the earplug is custom formed for a user.

13. The earplug according to claim 11, where the material is silicone.

14. The earplug according to claim 11, where the lubricant is mineral oil.

15. The earplug according to claim 11, where the lubricant has a specific gravity between 0.7 to 0.9.

16. The earplug according to claim 11, where the lubricant has a kinematic viscosity at 40 degrees C. between 10 to 20 mm̂2/s.

17. The earplug according to claim 11 where the mass of the lubricant absorbed after the treatment time is between 0.1 g to 1.5 g.

18. The earplug from claim 17, where the mass of the lubricant absorbed after the treatment time is between 0.3 g to 0.5 g.

19. A self lubricating earplug comprising:
a cured silicone plug, where the plug is configured to fit in an ear canal ; and
a lubricant which permeates the plug, where the permeated plug is configured to seep lubricant while inserted into the ear canal.

20. The earplug according to claim 19, where the lubricant has a permeated mass within about 0.1 g to 1.5 g, where the lubricant has a specific gravity between about 0.7 to 0.9, and where the lubricant has a kinematic viscosity at 40 degrees C. between about 10 to 20 mm̂2/s.
